(19) United States
(12) Patent Application Publication
(10) Pub. No.: US 2005/0175999 A1
(43) Pub. Date: Aug. 11, 2005

(54) Methods for determining binding affinities

(76) Inventors: Scott L. Klakamp, Fremont, CA (US); David G. Myszka, Salt Lake City, UT (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/488,371

(22) PCT Filed: Aug. 30, 2002
(86) PCT No.: PCT/US02/28163

Related U.S. Application Data
(60) Provisional application No. 60/316,147, filed on Aug. 30, 2001.
(60) Provisional application No. 60/355,534, filed on Feb. 8, 2002.

(51) Int. Cl. C12Q 1/68; G06F 19/00; G01N 33/48; G01N 33/50; G01N 33/543

(52) U.S. Cl. 435/6; 436/518; 702/19

(57) ABSTRACT

The present invention relates generally to methods for screening a plurality of ligands using a biosensor device. More particularly, the present invention relates to methods for screening a plurality of antibodies from complex solutions using a surface plasmon resonance device. The methods of this invention provide kinetic and equilibrium information for such screening assays. The present invention also relates to systems for determining kinetic rate constants for such screening assays.

### TECHNICAL FIELD OF THE INVENTION

The present invention relates generally to methods for screening a plurality of ligands using a biosensor device. More particularly, the present invention relates to methods for screening a plurality of antibodies from complex solutions using a surface plasmon resonance device. The methods of this invention provide kinetic and equilibrium information for such screening assays. The present invention also relates to systems for determining kinetic rate constants for such screening assays.

### BACKGROUND OF THE INVENTION

With the advent of combinatorial libraries, there is an increasing need for developing a method for screening a plurality of ligands that enables the rapid and efficient determination of binding affinities.

One such need is for screening methods for determining accurate kinetic and binding information for ligands in a complex solution, i.e., a solution containing an unpurified ligand. In complex solutions, the ligand concentration is unknown. Screening methods in complex solutions therefore cannot determine the kinetic association rate constant, kₐ, without a known ligand concentration. In the absence of kₐ, the binding affinity also cannot be determined. Consequently, current screening methods in complex solutions are limited to providing only qualitative information on the presence of, or relative binding affinity of, a specific ligand in the complex solution. If accurate kinetic and binding information are to be determined, the screening method requires purified ligand. The requirement for using purified ligands for kinetic characterization makes current methods time consuming and expensive.

Another need is for screening methods for determining accurate kinetic and binding information for polyvalent ligands, such as antibodies. In the case of antibodies, a generally accepted paradigm is that antibody binding affinity is determined by the dissociation rate constant, k_{d}, and that kₐ does not vary from one antibody to another. According to this paradigm, antibody binding kinetics typically rely only on k_{d}. Such misleading binding information makes the identification of useful antibodies more difficult.

The present invention meets the needs referred to above by providing a screening method for determining kinetic and binding information for interactions between a ligand and its binding partner using a biosensor device. The present invention also provides a method for determining such information for ligands in a complex solution. The present invention further provides a method for screening polyvalent ligands.

### SUMMARY OF THE INVENTION

The invention relates to a method for screening a plurality of ligands using a biosensor device. The invention also relates to methods for determining kinetic and equilibrium information for a plurality of ligand-binding partner interactions. The invention further relates to systems for determining kinetic rate constants for a plurality of ligand-binding partner interactions.

In some embodiments, the invention provides a method for screening a plurality of ligands using a biosensor device, comprising the steps of (a) contacting a biorecognition surface comprising a ligand of interest with a solution containing a binding partner; (b) collecting data for binding of the binding partner to the ligand; (c) globally fitting the data to a maximum response determined for a plurality of ligands binding to the binding partner and locally fitting the data to determine kinetic rate constants; and (d) calculating a binding affinity from the kinetic rate constants. In some embodiments, the biorecognition surface is prepared by ligand capture from the screening solution. In some embodiments, the ligand of interest is selected from the group consisting of proteins, including, but not limited to, antibodies, receptors and enzymes; nucleic acids; carbohydrates; lipids; and small molecules. In some embodiments, the binding partner is selected from the group consisting of proteins, including, but not limited to, antigens, receptors and enzymes; nucleic acids; carbohydrates; lipids; and small molecules. In some embodiments, the biosensor device is selected from the group consisting of an evanescent wave, total internal reflection fluorescence and surface plasmon resonance devices.

In some embodiments, the invention provides a method for screening a plurality of ligands from a complex solution using a biosensor device, comprising the steps of (a) contacting a biorecognition surface comprising a ligand of interest with solution containing a binding partner, wherein the biorecognition surface is prepared by ligand capture from the complex solution; (b) collecting data for binding of the binding partner to the ligand; (c) globally fitting the data to a maximum response determined for a plurality of ligands binding to the binding partner and locally fitting the data to determine kinetic rate constants; and (d) calculating a binding affinity from the kinetic rate constants. In some embodiments, the ligand of interest is selected from the group consisting of proteins, including, but not limited to, antibodies, receptors and enzymes; nucleic acids; carbohydrates; lipids; and small molecules. In some embodiments, the binding partner is selected from the group consisting of proteins, including, but not limited to, antigens, receptors and enzymes; nucleic acids; carbohydrates; lipids; and small molecules. In some embodiments, the biosensor device is selected from the group consisting of an evanescent wave, total internal reflection fluorescence and surface plasmon resonance devices.

In some embodiments, the invention provides a method for screening a plurality of antibodies from complex solutions using a surface plasmon resonance device, comprising the steps of (a) contacting a biorecognition surface comprising an antibody with solution containing an antigen, wherein the biorecognition surface is prepared by antibody capture from the complex solution; (b) collecting data for binding of the antigen to the antibody; (c) globally fitting the data to a maximum response determined for a plurality of antibodies binding to the antigen and locally fitting the data to determine kinetic rate constants; and (d) calculating a binding affinity from the kinetic rate constants.

In some embodiments, the invention provides a method for determining kinetic rate constants for a plurality of ligand-binding partner interactions using a biosensor device, comprising the steps of (a) contacting a biorecognition surface comprising the ligand with a solution containing the binding partner; (b) collecting data for binding of the binding partner to the ligand; and (c) globally fitting the data to a maximum response determined for a plurality of ligands binding to the binding partner and locally fitting the data to determine kinetic rate constants. In some embodiments, the ligand is selected from the group consisting of proteins, including, but not limited to, antibodies, receptors and enzymes; nucleic acids; carbohydrates; lipids; and small molecules. In some embodiments, the binding partner is selected from the group consisting of proteins, including, but not limited to, antigens, receptors and enzymes; nucleic acids; carbohydrates; lipids; and small molecules. In some embodiments, the biosensor device is selected from the group consisting of an evanescent wave, total internal reflection fluorescence and surface plasmon resonance devices.

In some embodiments, the invention provides a method for determining kinetic rate constants for a plurality of antibody-antigen interactions using a biosensor device, comprising the steps of (a) contacting a biorecognition surface comprising an antibody with a solution containing the antigen; (b) collecting data for binding of the antigen to the antibody; and (c) globally fitting the data to a maximum response determined for a plurality of antibodies binding to the antigen and locally fitting the data to determine kinetic rate constants. In some embodiments, the biosensor device is selected from the group consisting of an evanescent wave, total internal reflection fluorescence and surface plasmon resonance devices. In some embodiments, the antibody capture is from a complex solution. In some embodiments, the antibody capture is from a pure solution.

In some embodiments, the invention provides a system for determining kinetic rate constants for a plurality of ligand-binding partner interactions using a biosensor device, comprising (a) a biorecognition surface comprising a ligand; (b) a means for processing data for binding interactions between the ligand and the binding partner; and (c) a means for globally fitting the data to a maximum response determined for a plurality of ligands binding to the binding partner and locally fitting the data to determine the rate constants. In some embodiments, the biorecognition surface is prepared by ligand capture. In some embodiments, the biorecognition system is prepared by ligand capture from a complex solution. In some embodiments, the biorecognition system is prepared by ligand capture from a pure solution. In some embodiments, the ligand is selected from the group consisting of proteins, including, but not limited to, antibodies, receptors and enzymes; nucleic acids; carbohydrates; lipids; and small molecules. In some embodiments, the binding partner is selected from the group consisting of antigens, proteins, including, but not limited to, antigens, receptors and enzymes; nucleic acids; carbohydrates; lipids; and small molecules. In some embodiments, the biosensor device is selected from the group consisting of an evanescent wave, total internal reflection fluorescence and surface plasmon resonance devices.

In some embodiments, the invention provides a system for determining kinetic rate constants for a plurality of antibody-antigen interactions using a biosensor device, comprising (a) a biorecognition surface comprising an antibody; (b) a means for processing data for binding interactions between an antigen and the antibody; and (c) a means for globally fitting the data to a maximum response determined for a plurality of antibodies binding to the antigen and locally fitting the data to determine the rate constants. In some embodiments, the biorecognition system is prepared by antibody capture from a complex solution. In some embodiments, the biorecognition system is prepared by antibody capture from a pure solution. In some embodiments, the biosensor device is selected from the group consisting of an evanescent wave, total internal reflection fluorescence and surface plasmon resonance devices.

### BRIEF DESCRIPTION OF THE DRAWING FIGURES

FIG. 1 shows a typical set of sensorgrams for capturing antibody to a protein A immobilized surface and for binding of antigen to the antibody-captured protein A surface. Antibody was captured by an immobilized protein A surface (A). The antibody-captured protein A surface was washed for 10 minutes to stabilize the baseline signal (B). A buffer injection was collected to gather information about the background surface decay (C). Finally, a single concentration of antigen was injected over the surface (D). One protein A surface served as the control (data shown were reference subtracted). The box indicates the antibody capture level at the time of antigen injection.

FIG. 2 shows a typical set of sensorgrams for normalizing the background decay of an antibody-captured protein A surface.

FIG. 3 shows typical raw and normalized sensorgrams of antigen binding to antibody-captured protein A surface.

FIG. 4 shows global analysis of normalized sensorgrams.

FIG. 5 shows a typical set of sensorgrams of antigen binding to antibody-captured protein A surfaces in a high throughput screen.

FIG. 6 shows a plot of antibody capture level versus observed antigen binding response.

FIG. 7 shows a typical set of sensorgrams of antigen binding to antibody-captured protein A surfaces using a range of antigen concentrations.

FIG. 8 shows a plot of antibody affinities determined from single or multiple concentrations of antigen.

FIG. 9 shows the capture-coupling method for immobilizing antibody to a protein A surface.

FIG. 10 shows normalized data for antigen binding to antibody immobilized using the capture-coupling method.

### DETAILED DESCRIPTION

Definitions

Unless defined otherwise, all technical and scientific terms used herein have the same meaning as commonly understood by one of ordinary skill in the art to which this invention belongs. In case of conflict, the present application, including the definitions, will control. Also, unless otherwise required by context, singular terms shall include pluralities and plural terms shall include the singular. All publications, patents and other references mentioned herein are incorporated by reference.

Although methods and materials similar or equivalent to those described herein can be used in practice or testing of the present invention, exemplary suitable methods and materials are described below. The materials, methods and examples are illustrative only, and are not intended to be limiting. Other features and advantages of the invention will be apparent from the detailed description and from the claims.

Throughout the specification and claims, the word “comprise,” or variations such as “comprises” or “comprising,” will be understood to imply the inclusion of a stated integer or group of integers but not the exclusion of any other integer or group of integers.

In order to further define this invention, the following terms and definitions are herein provided.

As used herein, the term “biosensor device” means an analytical device comprising a biorecognition surface. Such a device typically produces a signal in response to a binding interaction at the biorecognition surface. The term includes, but is not limited to, evanescent wave, total internal reflection fluorescence (“TIRF”) and surface plasmon resonance (“SPR”) devices.

As used herein, the term “biorecognition surface” means a solid support comprising a ligand of interest.

As used herein, the term “solid support” means a material in the solid-phase that interacts with reagents in the liquid phase by heterogeneous reactions. Solid-supports can be derivatized with ligands by covalent or non-covalent bonding through one or more attachment sites, thereby “immobilizing” the ligand to the solid-support. The term includes, but is not limited to, glass surfaces, metal-coated glass surfaces, such as gold-coated, and modifications thereof. Suitable modifications include, but are not limited to, interactive surface layers. Examples of interactive surface layers include, but are not limited to, carboxymethyl-dextran hydrogel, alkoxy silanes (e.g., BIO-CONEXT™ from United Chemical Technologies, Inc.) and self-assembled monolayers (“SAMs”).

As used herein, the term “complex solution” means a solution comprising an unpurified ligand of interest. The term includes, but is not limited to, cell culture media, hybridoma supernatants, ascites fluid, serum, cell lysates or fractions thereof, column effluents, mixtures of ligand with other substances and the like.

As used herein, the term “unpurified ligand” means a ligand with less than about 90% purity.

As used herein, the term a “pure solution” means a solution with greater than about 90% purity.

As used herein, the term “ligand capture” means the process by which an agent immobilized on a solid support (“a capture agent”) captures any ligand present in a solution. The term includes, but is not limited to, antibody capture. Capture agents include, but are not limited to, protein A and antibodies, such as anti-isotype antibodies.

As used herein, the term “sensorgram” means a plot of response (measured in “resonance units” or “RU”) as a function of time. The response corresponds to the amount of material that binds to a sensor surface. An increase of 1000 RU corresponds to an increase of mass on the sensor surface of approximately 1 ng/mm². “Rₘₐₓ” means the response corresponding to the maximum binding capacity of the sensor surface.

As used herein, the term “association” means the step where ligand bound to a sensor surface interacts with a binding partner in solution. This step is indicated on the sensorgram by an increase in RU as the binding partner binds to the surface-bound ligand.

As used herein, the term “dissociation” means the step where the flow of binding partner is replaced by, for example, a flow of buffer. This step is indicted on the sensorgram by a decrease in RU over time as binding partner dissociates from the surface-bound ligand.

As used herein, the terms “ligand of interest” and “binding partner” mean members of a specific binding pair. Examples of ligands include, but are not limited to, proteins, including, but not limited to, antibodies, receptors and enzymes; nucleic acids; carbohydrates; lipids; and small molecules. Examples of binding partners include, but are not limited to, proteins, including, but not limited to, antigens, receptors and enzymes; nucleic acids; carbohydrates; lipids; and small molecules.

As used herein, the term “antibody” means an intact immunoglobulin or a functional binding fragment thereof. Antibodies of this invention can be of any isotype or class (e.g., M, D, G, E and A) or any subclass (e.g., G1-4, A1-2) and can have either a kappa (κ) or lambda (λ) light chain.

As used herein, the term “F_{c}” means a portion of the heavy chain constant region of an antibody that is produced by papain digestion.

As used herein, the term “antigen” means a molecule containing one or more epitopes that will stimulate a host's immune system to make a humoral and/or cellular antigen-specific response.

As used herein, the term “epitope” means the site on an antigen to which a specific antibody molecule binds.

The following abbreviations are also used herein SPR, surface plasmon resonance; TIRF, total internal reflection fluorescence; CM-dextran, carboxymethyl-dextran; kₐ, association rate constant; k_{d}, dissociation rate constant; RU, response units; and SAMs, self-assembled monolayers.

Methods for Screening Solutions

In one aspect, the present invention provides methods for the rapid and efficient screening of a plurality of ligand samples using a biosensor device to determine intrinsic kinetic and binding information for ligand-binding partner interactions. The number of samples can be any number from 1 up to the limits of the biosensor devise, i.e., at least 10, at least 30, at least 50, at least 75, at least 100, at least 125, at least 150, at least 175, etc.

The methods of the invention can be utilized with any ligand. The ligand may be monovalent, divalent or polyvalent. Exemplary ligands that can be used in the methods of the invention include, but are not limited to, proteins, including, but not limited to, antibodies, receptors and enzymes; nucleic acids; carbohydrates; lipids; and small molecules. Similarly, the methods of the invention can be used with any binding partner. The binding partner can be monovalent, bivalent or polyvalent. Exemplary binding partners that can be used in the methods of the invention include, but are not limited to, proteins, including, but not limited to, antigens, receptors and enzymes; nucleic acids; carbohydrates; lipids; and small molecules.

In some embodiments, the ligand is purified. In other embodiments the ligand is unpurified. In some embodiments, the unpurified ligand is in a complex solution. Exemplary complex solutions are cell culture media, hybridoma supernatants, ascites fluid, serum, cell lysates or fractions thereof, column effluents, mixtures of ligand with other substances and the like.

In a preferred embodiment, the ligand is an antibody and the binding partner is an antigen. In some embodiments, the antibody is purified. In other embodiments, the antibody is unpurified. In some embodiments, the unpurified antibody is in a complex solution, such as (but not limited to), cell culture media, hybridoma supernatants, ascites fluid, serum, cell lysates or fractions thereof, column effluents, mixtures of ligand with other substances and the like.

Any suitable solid support can be used to generate a biorecognition surface for use in a biosensor device. In a preferred embodiment, the solid support is glass. In some embodiments, the solid support is gold-coated glass. Preferably the solid support is coated with an interactive surface layer. Exemplary interactive surface layers are carboxymethyl-dextran hydrogel, alkoxy silanes and self-assembled monolayers (“SAMs”).

In a preferred embodiment, the biosensor device is an SPR device, such as a BIACORE device.

In some embodiments, the ligand is immobilized directly to the interactive layer, such as by amine coupling to carboxymethyl-dextran. However, where a large number of samples are to be analyzed, immobilization of the ligand to a composition that provides an easily regeneratable surface is preferred. In this way, the biorecognition surface can be quickly and easily regenerated for repeated use with numerous samples.

In embodiments using unpurified ligand samples, particularly where the ligand is in a complex solution, a capturing agent is used to immobilize the ligand onto the surface of a solid support to generate a biorecognition surface. The choices of capture agent for a ligand of interest are well-known in the art. The selection of an appropriate capture agent for use in the methods of the invention is well within the skill of the art.

In a preferred embodiment, the samples are antibodies in hybridoma supernatant. If the sample contains an IgG antibody, for example, Protein A or an anti-IgG antibody can be used as a capture agent. Capture agents for other antibody isotypes are well known in the art, e.g., anti-isotype antibodies. Immobilizing the antibody on the solid surface using a capturing agent, in addition to permitting the screening of unpurified antibodies, provides a number of additional advantages including providing antibodies immobilized in a more homogeneous orientation and thus providing more uniform biorecognition surfaces than is provided by direct immobilization, and permitting rapid regeneration of the solid surface between batches of samples. Further, by immobilizing the antibody, one can utilize known concentrations of antigen in the binding step. Because the antigen concentration is known, the association rate (kₐ) can be determined. As a result, one can determine a more accurate binding affinity, i.e., one based on both association and dissociation rates, for each antibody.

According to the method, the biorecognition surface comprising the ligand is contacted with a single concentration of binding partner solution and the biosensor device collects data for the binding interaction between the ligand and the binding partner. In some embodiments, the biosensor device is an SPR device, the ligand is a purified ligand that is directly immobilized on a solid support. In some embodiments, the biosensor device is an SPR device, the ligand is a purified ligand that is captured by a capture agent immobilized on a solid support. In either of these embodiments, the ligand can be an antibody.

Contacting each sample with the same single concentration of binding partner instead of contacting each sample with a range of different concentrations allows the rapid and efficient analysis of large numbers of samples.

According to the methods of the invention, the collected binding data is then processed to correct the binding signal for general noise, non-specific binding of the binding partner to the solid support and baseline drift.

In the methods of the invention, kinetic analyses are performed by globally fitting the processed binding data. In the global fitting, the binding data from multiple samples is fit to a single binding-site model and a single “global” Rmax is determined. According to the global fitting analysis, kₐ and k_{d} are permitted to be “local” parameters that are determined using a constant Rmax, i.e., the global Rmax. Binding affinity is then determined for each sample using the kinetic rate constants.

As will be readily understood, the method according to this aspect of the invention is well suited for screening large collections of ligands. High binding affinity ligands identified by this method may be further evaluated in higher resolution experiments, e.g., in experiments utilizing multiple concentrations of binding partners.

System for Determining Kinetic Rate Constants for Ligand-Binding Partner Interactions

In a further aspect, the present invention provides a system for determining kinetic rate constants for ligand-binding partner interactions using a biosensor device. The system comprises (a) a biorecognition surface comprising a ligand; (b) a means for processing data for binding interactions between the ligand and a binding partner; and (c) a means for globally fitting the data to a maximum response determined for a plurality of ligand binding to the binding partner and locally fitting the data to determine the rate constants.

In some embodiments, the biosensor device is an SPR device. In some embodiments, the biosensor device is an evanescent wave device. In some embodiments, the biosensor device is a TIRF device.

In order that this invention may be better understood, the following examples are set forth. These examples are for purposes of illustration only and are not to be construed as limiting the scope of the invention in any manner.

### EXAMPLES

In these experiments, we screened a panel of monoclonal antibody supernatants to determine the binding affinities of the antibodies using SPR.

### Example 1

### Preparation of Biorecognition Surfaces

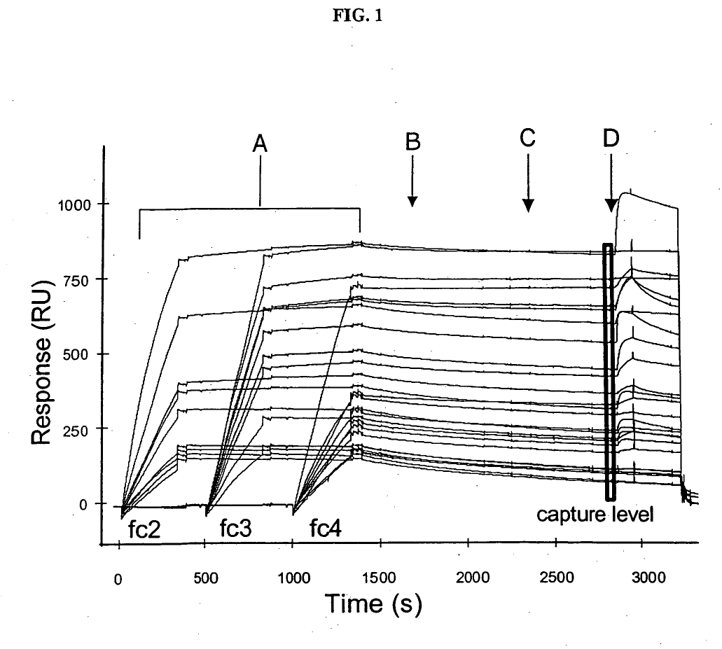

We prepared a biorecognition surface by first immobilizing Immunopure® Protein A (Pierce, Rockford, Ill.) to CM5 sensor chips (BIACORE AB, Uppsala, Sweden) in a BIACORE 2000 or 3000 instrument (FIG. 1) as follows.

In the BIACORE instrument, the sensor chips were pre-conditioned in water at a flow rate of 100 μL/min by applying two consecutive 20-μL pulses of 50 mM NaOH, 0.1% HCl (v/v) and 0.1% SDS. The individual flow cells were equilibrated with 10 mM HEPES buffer containing 150 mM NaCl and 0.005% P-20 Surfactant (BIACORE AB), pH 7.4 (“HBSP running buffer”) at a flow rate of 20 μL/min. Next, a solution of 70 μL of 50 mM N-hydroxysuccinimide (“NHS;” BIACORE AB) and 70 μL of 200 mM 1-(3-dimethylaminopropyl)-ethylcarbodiimide hydrochloride (“EDC;” BIACORE AB) was injected over the flow cells to activate the CM-dextran. Johnsson et al., Anal Biochem, 198, pp. 268-277 (1991). A solution of protein A (reconstituted in water to 5 mg/mL and diluted to 150 μg/mL in 10 mM sodium acetate at pH 5.0) was flowed across the flow cells for 7 min at a flow rate of 20 μL/min followed by a 140 μL injection of 1 M sodium ethanolamine.HCl at pH 8.5 (BIACORE AB). The immobilized protein A surfaces were immediately conditioned by three injections of 100 mM H₃PO₄ for 6 sec. Johnsson et al., Biotechniques, 11, pp. 620-627 (1991). The typical immobilization level of protein A was 6,000 to 8,000 RU.

In other experiments, we immobilized goat anti-IgG (Fc sp.) following the procedure described above for immobilizing protein A. In this procedure, we prepared a solution of IgG in 10 mM sodium acetate at pH 5.0 (100 μg/mL). The typical immobilization level of goat anti-IgG (Fc sp.) was 3,500 to 4,500 RU.

### Example 2

### Ligand Capture

We used the protein A or goat anti-IgG (Fc sp.) immobilized on the sensor chip surface to capture monoclonal antibodies from hybridoma supernatants (FIG. 1A) as follows. Myszka, J. Mol. Recognit., 12, pp. 279-284 (1999) and Svensson et al., Eur. J. Biochem., 258, pp. 890-896 (1998). Buffer (10 mM HEPES, 150 mM NaCl, 0.005% polysorbate-20, pH 7.4) containing 12 mg/mL of each BSA (Sigma, St. Louis, Mo.) and soluble carboxymethyl-dextran (“CM-dextran;” Fluka BioChemika) was flowed across the individual flow cells at a flow rate of 100 μL/min. Three hybridoma supernatant solutions containing antibodies of interest were diluted 1/25 in the same buffer and then separately injected over three flow cells for 5 min at a flow rate of 50 μL/min. The fourth flow cell was not exposed to an antibody solution and therefore, served as a control. The antibody-captured protein A surface was then washed for 10 min at a flow rate of 50 μL/min to remove any non-specific components adhering to the surface (FIG. 1B).

### Example 3

### Screening of Binding Partner

We screened the antibody-captured protein A surfaces for binding to antigen (FIG. 1D) as follows. Prior to antigen injection, a solution of buffer was injected to determine baseline drift caused by the decay of the antibody-captured protein A surface (FIG. 1C). Antigen binding was measured by flowing an antigen at a predetermined concentration across the individual flow cells for 1 min at a flow rate of 100 μL/min and then reintroducing the buffer for 5 min to initiate dissociation. After dissociation, the protein A surface was regenerated by injecting 10 μL of 100 mM H₃PO₄ for 12 sec. After regeneration, we repeated the antibody capture procedure described in Example 2 using three supernatants from the panel at a time until the entire panel was screened.

To determine the concentration of antigen to be used in the antigen-binding step, we first assessed the quality of antigen binding to the antibody-captured protein A surfaces using three different concentrations of antigen and from 3-6 hybridoma supernatants. An antigen concentration was chosen that resulted in a response having a minor to significant curvature in the association phases for the tested hybridoma supernatant solutions. This variation in curvature indicates either a range of association rates for the different antibodies or a concentration dependence of antigen binding to the same antibody.

When the response was asymptotic to the theoretical response level (calculated by multiplying the mass ratio of antigen to antibody by RU_{captured}), we repeated the antigen binding cycle using an antigen concentration diluted 3-fold (using further dilutions if necessary).

When the response had a change in RU less than 10 (i.e., not discernable from noise), we repeated the capture-binding cycle using a five-fold or more higher concentration of hybridoma supernatant solution. By increasing the antibody concentration, we increased the amount of antibody captured by the protein A surface and, thus, the amount of antigen bound.

### Example 4

### Data Processing

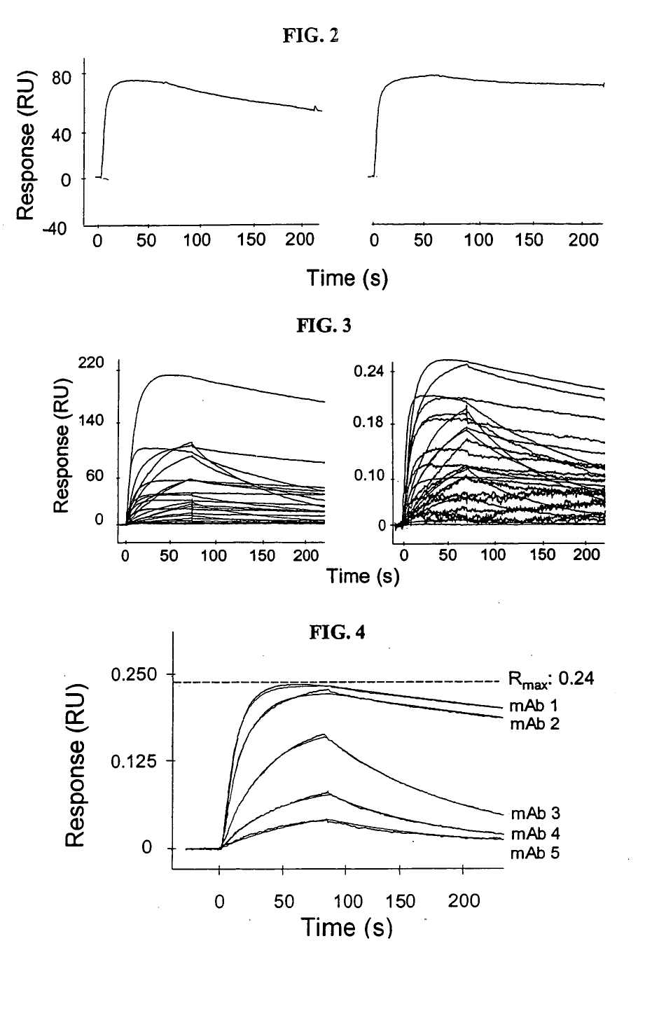

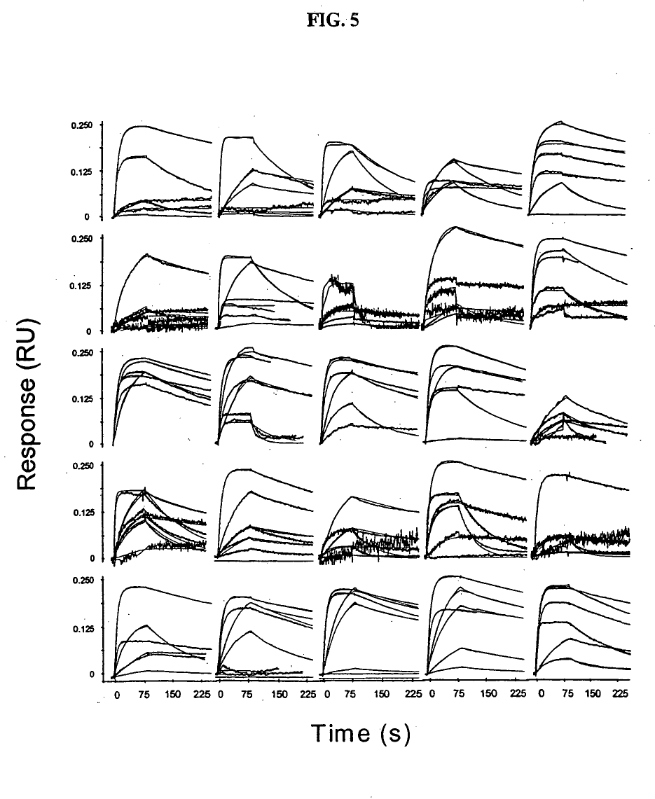

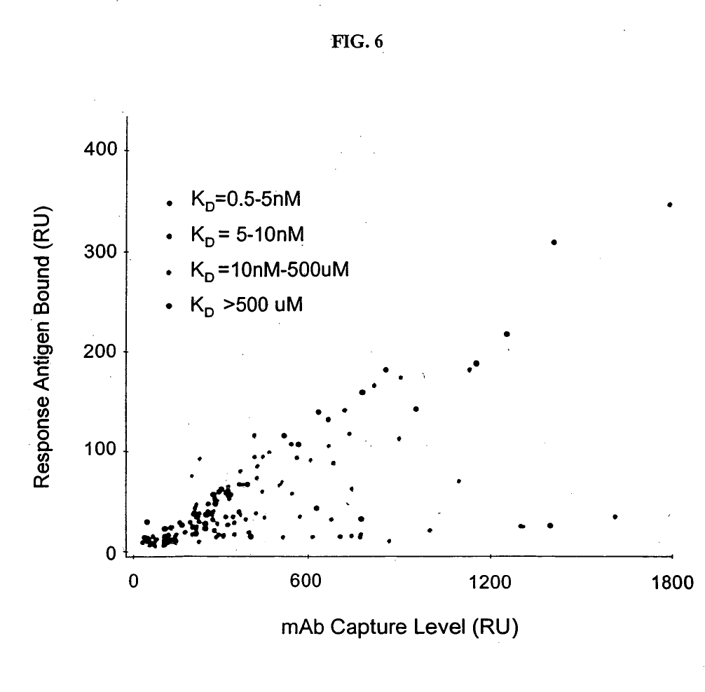

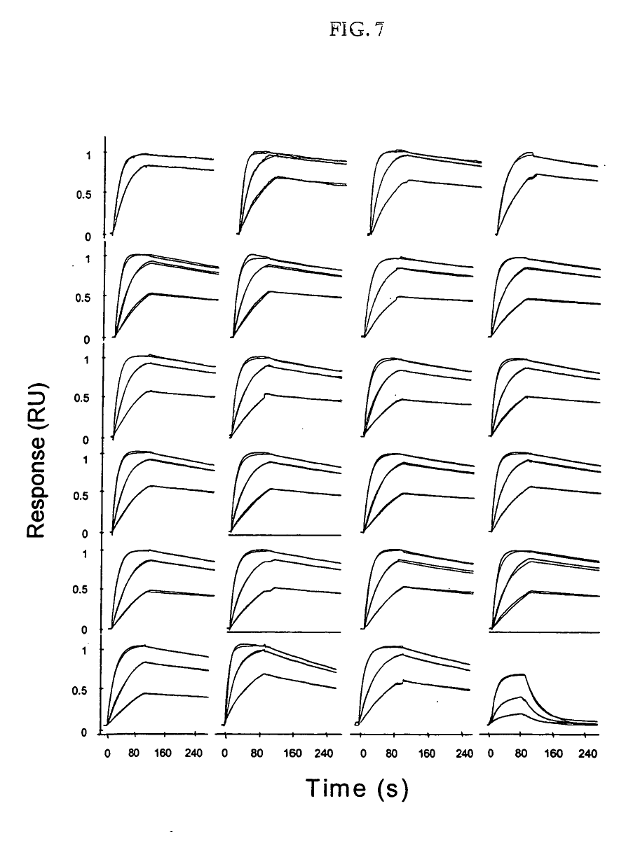

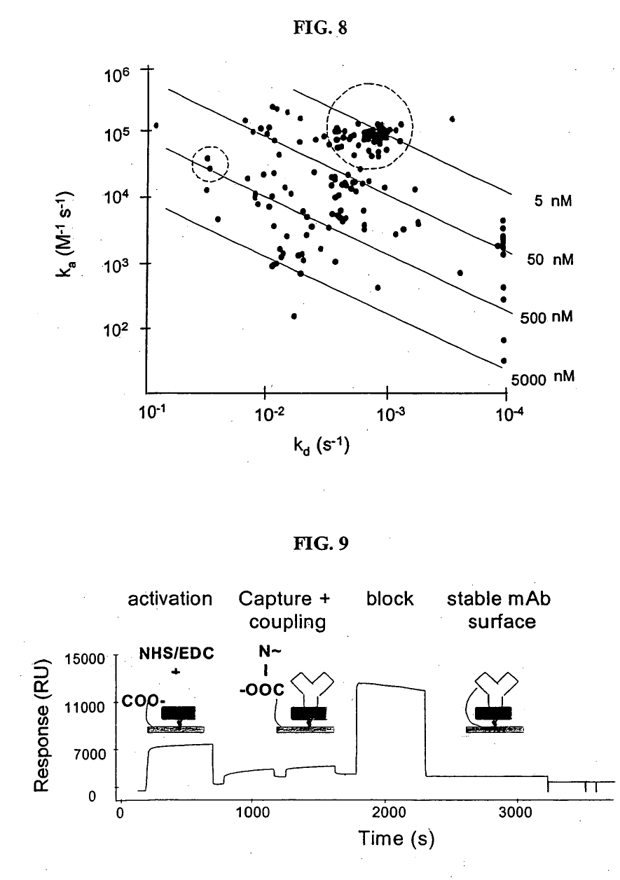

When sensorgrams for all members of the panel were obtained, we processed the data from the sensorgrams to correct the binding signal for general noise, non-specific binding of antigen to the protein A surface and baseline drift as follows (FIG. 2 and FIG. 3).

We first corrected the binding signal for bulk refractive index changes and non-specific binding by subtracting the signal for antigen flowed across the control flow cell which had a protein A immobilized surface but no antibody (“the control signal”) from the signal for antigen flowed across antibody-captured protein A surface.

Next, we aligned the signals and subtracted blank buffer injections to account for baseline drift (i.e., background decay of the antibody-captured protein A surface) (FIG. 2). Baseline drift was accounted for by subtracting the signal for the blank buffer injection, indicated by FIG. 1C, from that for the antigen injection, indicated by FIG. 1D.

Alternatively, we followed a modified protocol for subtracting baseline drift. In this protocol, an antigen binding signal (“signal 1”) was obtained as described above, but omitting the buffer injection preceeding the antigen injection. The protein A surface was then regenerated. Next, the antibody-captured protein A surface was prepared again following the procedure described above and a signal (“signal 2”) was obtained by flowing buffer alone across the surface for the remainder of the cycle. Baseline drift was accounted for by subtracting signal 2 from signal 1.

Lastly, we normalized the data with respect to the amount of antibody captured (FIG. 3). The normalization step consists of: (1) determining the antibody capture signal by averaging the signal obtained during the 20 seconds prior to antigen injection (indicated with a box in FIG. 1); (2) dividing the antigen binding signal by the antibody capture signal; and (3) multiplying the quotient by the mass ratio of antibody to antigen. These processing steps may be performed using BIACORE's Biaevaluations software.

### Example 5

### Globally Fitting the Binding Data

We determined the binding affinities for each of the antibodies in the panel. First, we performed kinetic analyses by globally fitting the binding data. Using a 1:1 binding model in the software program CLAMP (www.cores.utah.edu/interaction), we simultaneously fit six normalized sensorgrams for the binding of a single antigen to different antibody-captured protein A surfaces. Myszka et al., Trends Biochem. Sci., 23, pp. 149-150 (1998). We determined a global Rₘₐₓ value by first selecting six sensorgrams representing a range of antigen binding responses. Second, we globally fitted those sensorgrams holding Rₘₐₓ constant and locally fitted the association and dissociation rate constants. In subsequent fits, we applied the global Rₘₐₓ value and the fixed antigen concentration to locally fit the association and dissociation rate constants.

### Example 6

### Screening Analysis

We screened 150 mAbs from hybridoma supernatants in three panels for binding to an antigen following the protocols of Examples 1 through 5 (FIG. 5). Table 1 shows the kinetic rate constants for antibody-antigen interactions determined in the screen. We calculated the antigen binding affinities of the antibodies from the kₐ and k_{d} parameters from the processed sensorgrams.

### Example 7

### Medium Resolution Analysis

We tested the reliability of the screening procedures of Examples 1-6 by re-assaying 24 mAbs in the panel using a range of antigen concentrations for each Ab (“medium resolution analysis”) (FIG. 7). A 100 μL solution of supernatant containing an antibody of interest (diluted 25-fold in HBSP containing 100 mg/mL BSA) was flowed across a protein A surface at a flow rate of 20 μL/min. The antibody-captured protein A surface was washed for 6 min at a flow rate of 50 μL/min. Next, a 50 μL solution of antigen (0, 22.2, 66.6, 200.0 or 600.0 nM) was injected. The dissociation phase for most of the mAbs was monitored for 120 sec and the protein A surface was regenerated by a 10 μL injection of H₃PO₄ for most Abs. However, we found that, in the single antigenic concentration screen, six out of thirty mAbs from Panel 2 supernatants displayed dissociation rate constants of about 10⁻⁵ s⁻¹, giving rise to very stable Ab-Ag complexes. For these mAbs, we monitored the dissociation phase for 10 min in the medium resolution screen.

We processed the data as described in Example 4. We analyzed the processed data using a 1:1 interaction model in CLAMP. We normalized the data by dividing Rₘₐₓ calculated from the antigen-binding kinetics for each mAb. Table 1 shows the kinetic rate constants obtained in this medium resolution analysis.

TABLE 1
Kinetic parameters of Panels 1, 2, and 3 antigen-
antibody interactions from the low and medium-resolution analysis.

| | |
|---|---|
| Association rates | Dissociation rates |
| (M⁻¹ s⁻¹) | (s⁻¹) |

| | | | | |
|---|---|---|---|---|
| | Low- | Medium- | Low- | Medium- |
| Ab | resolution | resolution | resolution | resolution |

Supernatants Panel 1

| | | | | |
|---|---|---|---|---|
| 1 | 1.90E+05 | 3.06E+05 | 1.00E−05 | 2.50E−04 |
| 2 | 2.50E+05 | 2.51E+05 | 1.00E−03 | 6.46E−04 |
| 3 | 2.03E+05 | 2.01E+05 | 8.05E−04 | 7.12E−04 |
| 4 | 1.81E+05 | 2.48E+05 | 8.41E−04 | 9.20E−04 |
| 5 | 1.83E+05 | 2.26E+05 | 1.14E−03 | 9.79E−04 |
| 6 | 1.88E+05 | 1.96E+05 | 8.80E−04 | 9.63E−04 |
| 7 | 1.69E+05 | 1.33E+05 | 9.86E−04 | 6.62E−04 |
| 8 | 1.60E+05 | 1.63E+05 | 9.90E−04 | 8.49E−04 |
| 9 | 1.59E+05 | 1.52E+05 | 8.97E−04 | 8.41E−04 |
| 10 | 1.59E+05 | 1.64E+05 | 9.56E−04 | 9.67E−04 |
| 11 | 1.38E+05 | 1.50E+05 | 1.30E−03 | 8.94E−04 |
| 12 | 1.67E+05 | 1.67E+05 | 1.12E−03 | 1.04E−03 |
| 13 | 1.67E+05 | 1.62E+05 | 1.13E−03 | 1.02E−03 |
| 14 | 1.81E+05 | 1.68E+05 | 1.30E−03 | 1.07E−03 |
| 15 | 1.41E+05 | 1.66E+05 | 1.21E−03 | 1.06E−03 |
| 16 | 1.91E+05 | 1.51E+05 | 1.17E−03 | 9.85E−04 |
| 17 | 1.86E+05 | 1.53E+05 | 1.23E−03 | 1.07E−03 |
| 18 | 2.02E+05 | 1.35E+05 | 1.10E−03 | 9.76E−04 |
| 19 | 2.10E+05 | 1.46E+05 | 8.99E−04 | 1.06E−03 |
| 20 | 1.72E+05 | 1.19E+05 | 1.16E−03 | 9.58E−04 |
| 21 | 1.28E+05 | 9.46E+04 | 1.07E−03 | 8.56E−04 |
| 22 | 2.56E+05 | 2.19E+05 | 1.46E−03 | 2.14E−03 |
| 23 | 1.93E+05 | 1.56E+05 | 1.12E−03 | 1.68E−03 |
| 24 | 2.37e+05 | 6.86E+04 | 6.00E−02 | 2.30E−02 |

Supernatants Panel 2

| | | | | |
|---|---|---|---|---|
| 1 | 5.0 × 10⁵ | 8.6 × 10⁵ | 5.2 × 10⁻⁴ | 1.1 × 10⁻⁵ |
| 2 | 5.0 × 10⁵ | 5.0 × 10⁵ | 2.9 × 10⁻⁴ | 2.9 × 10⁻⁵ |
| 3 | 8.6 × 10⁵ | 9.5 × 10⁵ | 9.3 × 10⁻⁴ | 1.7 × 10⁻⁵ |
| 4 | 2.1 × 10⁵ | 2.1 × 10⁵ | 3.6 × 10⁻⁵ | 1.6 × 10⁻⁵ |
| 5 | 2.1 × 10⁵ | 1.9 × 10⁵ | 2.7 × 10⁻⁴ | 3.4 × 10⁻⁵ |
| 6 | 8.7 × 10⁵ | 5.0 × 10⁵ | 3.6 × 10⁻⁴ | 3.4 × 10⁻⁵ |

Supernatants Panel 3

| | | | | |
|---|---|---|---|---|
| 1 | 1.0E+07 | 7.3E+08 | 4.3E−04 | 5.9E−04 |
| 2 | 5.4E+06 | 1.5E+08 | 3.4E−04 | 3.3E−04 |
| 3 | 9.2E+06 | 4.1E+08 | 4.1E−04 | 1.4E−03 |
| 4 | 1.4E+07 | 3.7E+08 | 9.8E−04 | 1.3E−03 |
| 5 | 1.1E+07 | 4.2E+08 | 5.6E−04 | 1.8E−03 |
| 6 | 7.5E+07 | 1.1E+08 | 3.9E−03 | 5.8E−04 |
| 7 | 1.1E+07 | 3.7E+08 | 7.4E−04 | 2.2E−03 |
| 8 | 1.3E+07 | 1.5E+08 | 4.5E−04 | 1.1E−03 |
| 9 | 1.3E+07 | 2.6E+07 | 6.6E−04 | 3.7E−04 |

### Example 8

### Capture-Coupling

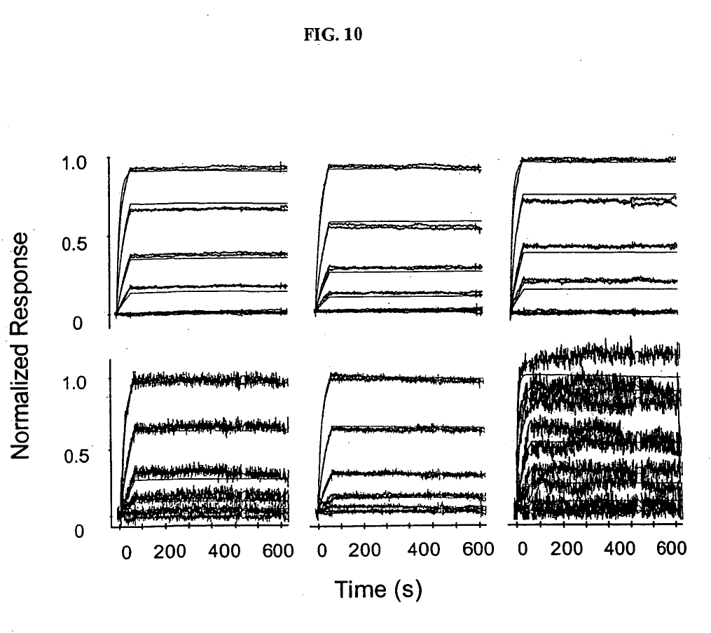

We also evaluated select, highly stable antigen-antibody interactions using antibody-coupled protein A surfaces (FIG. 9). A solution of NHS and EDC was injected over a protein A-CM-dextran surface for 7 min at a flow rate of 20 μL/min. A solution of antibody was flowed across the flow cells at a flow rate of 5-10 μL/min followed by an injection of 1 M sodium ethanolamine.HCl at pH 8.5. Antigen binding was measured by flowing a solution of antigen (0, 2.4, 7.4, 22.2, 66.7 or 200 nM) in HBSP containing 200 μg/mL BSA across the antibody-coupled protein A surface. The antibody-coupled protein A surfaces were regenerated using 10 mM H₃PO₄. We processed the data as described above and then fit the processed data to a 1:1 interaction model using CLAMP (FIG. 10). We determined kinetic rate constants and binding affinities.

1. A method for screening a plurality of ligands using a biosensor device, comprising the steps of:
a. contacting a biorecognition surface comprising a ligand of interest with a solution containing a binding partner;
b. collecting data for binding of the binding partner to the ligand;
c. globally fitting the data to a maximum response determined for a plurality of ligands binding to the binding partner and locally fitting the data to determine kinetic rate constants; and
d. calculating a binding affinity from the kinetic rate constants.

2. The method according to claim 1, wherein the biorecognition surface is prepared by ligand capture from the screening solution.

3. The method according to claim 1 or 2, wherein the ligand of interest is selected from the group consisting of proteins, antibodies, receptors, enzymes, nucleic acids, carbohydrates, lipids and small molecules.

4. The method according to claim 1 or 2, wherein the binding partner is selected from the group consisting of proteins, antigens, receptors, enzymes, nucleic acids, carbohydrates, lipids and small molecules.

5. The method according to claim 1 or 2, wherein the biosensor device is selected from the group consisting of an evanescent wave, total internal reflection fluorescence and surface plasmon resonance devices.

6. A method for screening a plurality of ligands from a complex solution using a biosensor device, comprising the steps of:
a. contacting a biorecognition surface comprising a ligand of interest with a solution containing a binding partner,
wherein the biorecognition surface is prepared by ligand capture from the complex solution;
b. collecting data for binding of the binding partner to the ligand;
c. globally fitting the data to a maximum response determined for a plurality of ligands binding to the binding partner and locally fitting the data to determine kinetic rate constants; and
d. calculating a binding affinity from the kinetic rate constants.

7. The method according to claim 6, wherein the ligand is selected from the group consisting of proteins, antibodies, receptors, enzymes, nucleic acids, carbohydrates, lipids and small molecules.

8. The method according to claim 6, wherein the binding partner is selected from the group consisting of proteins, antigens, receptors, enzymes, nucleic acids, carbohydrates, lipids and small molecules.

9. The method according to claim 6, wherein the biosensor device is selected from the group consisting of an evanescent wave, total internal reflection fluorescence and surface plasmon resonance devices.

10. A method for screening a plurality of antibodies from complex solutions using a surface plasmon resonance device, comprising the steps of:
a. contacting a biorecognition surface comprising antibody with a solution containing an antigen,
wherein the biorecognition surface is prepared by antibody capture from the complex solution;
b. collecting data for binding of the antigen to the antibody;
c. globally fitting the data to a maximum response determined for a plurality of antibodies binding to the antigen and locally fitting the data to determine kinetic rate constants; and
d. calculating a binding affinity from the kinetic rate constants.

11. A method for determining kinetic rate constants for a plurality ligand-binding partner interactions using a biosensor device, comprising the steps of:
a. contacting a biorecognition surface comprising the ligand with a solution containing the binding partner;
b. collecting data for binding of the binding partner to the ligand; and
c. globally fitting the data to a maximum response determined for a plurality of ligands binding to the binding partner and locally fitting the data to determine kinetic rate constants.

12. The method according to claim 11, wherein the ligand is selected from the group consisting of proteins, antibodies, receptors, enzymes, nucleic acids, carbohydrates, lipids and small molecules.

13. The method according to claim 11, wherein the binding partner is selected from the group consisting of proteins, antigens, receptors, enzymes, nucleic acids, carbohydrates, lipids and small molecules.

14. The method according to claim 11, wherein the biosensor device is selected from the group consisting of an evanescent wave, total internal reflection fluorescence and surface plasmon resonance devices.

15. A method for determining kinetic rate constants for a plurality of antibody-antigen interactions using a biosensor device, comprising the steps of:
a. contacting a biorecognition surface comprising an antibody with a solution containing the antigen;
b. collecting data for binding of the antigen to the antibody; and
c. globally fitting the data to a maximum response determined for a plurality of antibodies binding to the antigen and locally fitting the data to determine kinetic rate constants.

16. The method according to claim 15, wherein the biosensor device is selected from the group consisting of an evanescent wave, total internal reflection fluorescence and surface plasmon resonance devices.

17. The method according to claim 15, wherein the antibody capture is from a complex solution.

18. The method according to claim 15, wherein the antibody capture is from a pure solution.

19. A system for determining kinetic rate constants for a plurality of ligand-binding partner interactions using a biosensor device, comprising:
a. a biorecognition surface comprising a ligand;
b. a means for processing data for binding interactions between the ligand and the binding partner; and
c. a means for globally fitting the data to a maximum response determined for a plurality of ligands binding to the binding partner and locally fitting the data to determine the rate constants.

20. The system according to claim 19, wherein the biorecognition surface is prepared by ligand capture.

21. The system according to claim 20, wherein the ligand capture is from a complex solution.

22. The system according to claim 20, wherein the ligand capture is from a pure solution.

23. The system according to claim 19, wherein the biosensor device is selected from the group consisting of an evanescent wave, total internal reflection fluorescence and surface plasmon resonance devices.

24. A system for determining kinetic rate constants for a plurality of antibody-antigen interactions using a biosensor device, comprising:
a. a biorecognition surface comprising an antibody;
b. a means for processing data for binding interactions between an antigen and the antibody; and
c. a means for globally fitting the data to a maximum response determined for a plurality of antibodies binding to the antigen and locally fitting the data to determine the rate constants.

25. The system according to claim 24, wherein the biorecognition surface is prepared by antibody capture.

26. The system according to claim 25, wherein the antibody capture is from a complex solution.

27. The system according to claim 25, wherein the antibody capture is from a pure solution.

28. The system according to claim 24 or 25, wherein the biosensor device is selected from the group consisting of an evanescent wave, total internal reflection fluorescence and surface plasmon resonance devices.
